(19) United States
(12) Patent Application Publication
(10) Pub. No.: US 2021/0148882 A1
(43) Pub. Date: May 20, 2021

(54) METHOD AND SYSTEM FOR DETERMINING QUALITY OF A FUEL

(71) Applicant: BENNAMANN SERVICES LTD, Newquay, Cornwall (GB)

(72) Inventors: Thomas William BRADSHAW, Oxon (GB); Christopher Mark MANN, St. Mawgan, Cornwall (GB); Matthew Charles Seabrook HERITAGE, Abingdon, Oxon (GB)

(73) Assignee: BENNAMANN SERVICES LTD, Newquay, Cornwall (GB)

(21) Appl. No.: 17/059,140

(22) PCT Filed: May 29, 2018
(86) PCT No.: PCT/IB2018/053821

§ 371 Date: Nov. 25, 2020

(51) Int. Cl. G01N 33/22; G01N 29/024

(52) U.S. Cl. CPC G01N 33/225; G01N 2291/02809; G01N 29/024

(57) ABSTRACT

Systems and methods use sound waves for evaluating a fuel. The fuel supplied from a storage tank to an engine by a feed pipe can be evaluated by determining its properties based on the velocity of one or more sound waves in the fuel.

### FIELD OF THE DISCLOSURE

Aspects of this disclosure relate generally to a system and a method for evaluating the properties of a fluid or gas, and in particular, a fuel, including systems and methods for determining the quality of methane fuel for a fuel supply system of an engine or for detecting impurities in a bio-gas produced according to anaerobic digestion process.

### BACKGROUND

Methane is commonly used as a fuel, and may come from a variety of sources. For instance, as set forth in U.S. Pat. No. 9,927,067, titled “Liquid Methane Storage System and Method,” anaerobic digestion is a technique for converting organic matter into biogas, and ultimately, methane gas.

However, methane from some sources may be of variable quality. In addition, if a liquid methane tank is left unattended for an extended period of time, higher levels of hydrocarbons and impurities may accumulate in the tank, leading to problems with subsequent use of the tank. Moreover, when used a fuel, flow of a liquid or gas may be insufficient, interrupted, or otherwise incorrect. This may be true, for instance, with respect to the flow of a methane fuel.

Accordingly, there is a need for a system and method that can effectively evaluate the properties of, including in some instances the flow of, fluids and gases such as methane and biogas.

### SUMMARY

According to some embodiments, systems and methods are provided for determining the quality of methane fuel and its flow, for instance with respect to a fuel supply system of an engine, and/or for detecting impurities in methane-based a bio-gas produced according to an anaerobic digestion process. The determination can be made, for instance, using one or more sound waves. In certain aspects, the determination is based on the measured speed of one or more sound waves.

In some instances, poor quality fuel can lead to poor performance, such as sub-optimal combustion in an engine, and in some cases, may cause knocking and/or failure of an engine. According to some embodiments, systems and methods are provided that can detect the quality of a fuel supplied to the engine in a reliable manner and prevent such sub-optimal performance of an engine.

The present disclosure includes various embodiments of an apparatus for measuring the properties of a gas or fluid flowing in a feed pipe. In accordance with some embodiments, the apparatus may comprise a first transducer coupled to the feed pipe and configured to generate a first sound wave passing in a first direction through the gas or fluid flowing along the feed pipe. The apparatus may comprise a second transducer coupled to the feed pipe and configured to receive the first sound wave passing in the first direction through the gas or fluid flowing along the feed pipe. The apparatus may comprise timing-circuitry in electrical communication with the first and second transducers and configured to measure a velocity of the first sound wave passing in the first direction from the first transducer to the second transducer.

In some embodiments, the second transducer is configured to generate a second sound wave passing in a second direction through the gas or fluid flowing along the feed pipe, and the first transducer is configured to receive the second sound wave passing in the second direction through the gas or fluid flowing along the feed pipe. The timing-circuitry may be configured, for example, to measure a velocity of the second sound wave passing in the second direction from the second transducer to the first transducer. The timing-circuitry may also be configured to calculate an average sound wave velocity based on at least the measured velocities of the first sound wave and the second wave. In some embodiments, the timing-circuitry is configured to calculate a velocity of the gas or fluid in the feed pipe based on at least the measured velocities of the first sound wave and the second wave, and the apparatus may comprise processing-circuitry in electrical communication with the timing-circuitry and configured to determine a composition of the gas or fluid based on at least the average sound wave velocity. The processing-circuitry may be further configured to determine the rate of flow of the gas or fluid in the feed pipe based at least in part on the measured velocities of the first and second sound waves.

In some embodiments, the first and second transducers are disposed on an exterior surface of the feed pipe, and the first and second transducers are aligned with each other along a lateral axis of the feed pipe. In some embodiments, the first and second transducers are disposed within the feed pipe and are displaced from each other along a longitudinal axis of the feed pipe. In some embodiments, the first and second transducers are disposed on an exterior surface of the feed pipe, and the first and second transducers are displaced from each other along a longitudinal axis of the feed pipe. In certain aspects, the apparatus may use a first reflector disposed within the feed pipe and aligned with the first transducer and a second reflector disposed within the feed pipe and aligned with the second transducer, such that the first and second reflectors are configured to alter the direction of the first and second sound waves passing through the gas or fluid to align with the direction of gas or fluid flow. The first and second reflectors may be, for example, one or more mirrors.

The present disclosure includes various embodiments of a method for measuring flow properties of a gas or fluid flowing in a feed pipe. In accordance with some embodiments, the method may comprise a step (a) of generating, by a first transducer coupled to the feed pipe, a first sound wave to pass in a first direction through the gas or fluid flowing along the feed pipe; a step (b) of receiving, by a second transducer coupled to the feed pipe, the first sound wave passing in the first direction through the gas or fluid flowing along the feed pipe; and a step (c) of measuring, for example by timing-circuitry, a velocity of the first sound wave passing in the first direction from the first transducer to the second transducer.

In some embodiments, the method may further comprise a step (d) of generating, by the second transducer, a second sound wave to pass in a second direction through the gas or fluid flowing along the feed pipe; a step (e) of receiving, by the first transducer, the second sound wave passing in the second direction through the gas or fluid flowing along the feed pipe, and a step (f) of measuring, for example by the timing-circuitry, a velocity of the second sound wave passing in the second direction from the second transducer to the first transducer.

In some embodiments, the method may comprise, for instance after step (f), calculating, by the timing-circuitry, an average sound wave velocity based on at least the measured velocities of the first sound wave and the second wave. In some embodiments, the method may also comprise after step (f), calculating, by the timing-circuitry, a velocity of the gas or fluid flowing along the feed pipe based on at least the measured velocities of the first sound wave and the second wave. In some embodiments, the method uses processing-circuitry to determine a composition of the gas or fluid based on at least the average sound wave velocity.

The present disclosure includes various embodiments of a system for detecting impurities in a fuel, for instance, comprising a methane gas. The system may have a gas vessel, and the fuel may be transported within the gas vessel. The system may comprise a gas composition sensor configured to: (i) generate one or more sound waves passing through the fuel in the gas vessel, and (ii) measure a velocity of each sound wave passing through the fuel in the gas vessel.

In some embodiments, the system may comprise a controller in electrical communication with the gas composition sensor, wherein the controller is configured to: (i) receive from the gas composition sensor an indication of the velocity of each sound wave passing through the fuel, and (ii) determine a composition of the fuel based on at least the received indication. In some embodiments, the indication is a measurement signal. In some embodiments, the determining the composition of the fuel comprises determining the relative level of the methane in the fuel.

Additionally, the system may further comprise a storage tank configured to store the fuel. In some embodiments, the system may comprise an engine configured to operate on the fuel, wherein the gas vessel is a feed pipe configured to supply the fuel from the storage tank to the engine.

The present disclosure includes various embodiments of a method for detecting impurities in a fuel comprising a methane gas. The method may comprise a step of supplying, by a feed pipe, the fuel from a storage tank to an engine. The method may also comprise steps of generating, by a gas composition sensor, one or more sound waves to pass through the fuel flowing along the feed pipe to the engine and measuring, by the gas composition sensor, a velocity of each sound wave passing through the fuel flowing along the feed pipe to the engine. The method may comprise a step of receiving, by a controller, from the gas composition sensor a measurement signal indicating the velocity of each sound wave. The method may comprise a step of determining, by the controller, a level of the methane gas in the fuel based on at least the received measurement signal. In some embodiments, the method may comprise determining, by the controller, an approximate fraction of the methane gas in the fuel based on at least the received measurement signal.

In some embodiments, the method may comprise a step of controlling, by a flow control valve coupled to the feed pipe, a flow rate of the fuel flowing along the feed pipe to the engine based on the determined level of methane gas in the fuel. In some embodiments, the method may comprise the step of reducing, by a flow control valve coupled to the feed pipe, a flow rate of the fuel flowing along the feed pipe to the engine when the determined level of methane gas in the fuel is below a first predetermined threshold. In some embodiments, the first predetermined threshold is a methane number of 90.

In some embodiments, the method may comprise the step of shutting off, by a flow control valve coupled to the feed pipe, a flow of the fuel to the engine when the determined level of methane gas in the fuel is below a second predetermined threshold. In some embodiments, the first predetermined threshold is a first methane number, and the second predetermined threshold is a second methane number, and the second methane number is less than the first methane number.

The present disclosure includes various embodiments of a system for determining a composition of a bio-gas comprising a methane gas and a secondary material. The system may comprise an anaerobic digestion unit configured to produce the bio-gas according to an anaerobic digestion process. In some instance, the anaerobic digestion unit comprises one or more storage tanks configured to vent the bio-gas and a bio-gas outlet pipe configured to receive the bio-gas vented from the one or more storage tanks. The system may comprise a gas cleaning stage configured to remove at least a portion of the secondary material from the bio-gas produced in the anaerobic digestion unit, and wherein the gas cleaning stage comprises an inlet configured to receive the bio-gas from the bio-gas outlet pipe and an outlet port configured to supply a remaining portion of the bio-gas to a methane storage tank. The system may further comprise a gas composition sensor coupled to one or more of the inlet port and the outlet port of the gas cleaning stage, and wherein the gas composition sensor is configured to: (i) generate one or more sound waves passing through the bio-gas flowing along the gas outlet pipe or the outlet port, and (ii) measure a speed of each sound wave passing through the bio-gas flowing along the gas outlet pipe or the outlet port. In some embodiments, the system may comprise a controller in electrical communication with the gas composition sensor, wherein the controller is configured to: (i) receive from the gas composition sensor a measurement signal indicating the speed of each sound wave, and (ii) determine a level of the secondary material or a methane gas level in the bio-gas based on at least the received measurement signal. The secondary material may be, for example, carbon dioxide.

In some embodiments, the gas composition sensor is coupled to the outlet port. In some embodiments, the system may comprise a flow control valve that is configured to: (i) receive the remaining portion of the bio-gas from the outlet port, and (ii) direct the remaining portion of the bio-gas to the methane storage tank or back to the inlet port of the gas cleaning stage. For instance, bio-gas may be directed based on the level of the secondary material in the bio-gas. For example, in some embodiments, the controller is in electrical communication with the flow control valve and configured to transmit a command to the flow control valve to direct the remaining portion of the bio-gas back to the inlet of the gas cleaning stage when the fraction of methane gas in the remaining portion of the bio-gas is below a predetermined level.

The present disclosure includes various embodiments of a method for determining a composition of a bio-gas comprising a methane gas and a secondary material. The method may comprise a step of producing, by an anaerobic digestion unit, the bio-gas according to an anaerobic digestion process. The method may comprise a step of supplying, by a gas outlet pipe, the bio-gas gas to an inlet of a gas cleaning stage. The method may comprise a step of removing, by the gas cleaning stage, at least a portion of the secondary material from the bio-gas produced by the anaerobic digestion unit. The method may comprise a step of supplying, by an outlet port, a remaining portion of the bio-gas from the gas cleaning stage to a methane gas storage tank. The method may comprise a step of generating, by a gas composition sensor, one or more sound waves to pass through the remaining portion of the bio-gas flowing along the outlet port. The method may comprise a step of measuring, by the gas composition sensor, a velocity of each sound wave through the remaining portion of the bio-gas flowing along the outlet port. The method may comprise a step of receiving, by a controller, from the gas composition sensor a measurement signal indicating the velocity of each sound wave. The method may comprise a step of determining, by the controller, a level of the methane gas in the remaining portion of the bio-gas based on at least the received measurement signal. In some embodiments, the method may comprise determining, by the controller, an approximate fraction of the methane gas in the fuel based on at least the received measurement signal.

In some embodiments, the method may comprise redirecting, by a flow control valve coupled to the outlet port, a flow of the remaining portion of the bio-gas back to the inlet of the gas cleaning stage when the determined level of methane gas in the fuel is below a first predetermined threshold. In some embodiments, the method may comprise supplying, by an inlet pipe, a biological material to one or more storage tanks of the anaerobic digestion unit. In some embodiments, the step of producing includes using the biological material to produce the bio-gas and venting the bio-gas from the one or more storage tanks to the gas outlet pipe.

According to some embodiments, a monitoring system for reporting characteristics of a fuel for a vehicle is provided. The monitoring system may comprise, for instance, a gas composition sensor configured to: (i) generate one or more sound waves passing through the fuel, and (ii) measure a velocity of each sound wave passing through the fuel. The system may further comprise a controller in electrical communication with the gas composition sensor, wherein the controller is configured to: (i) receive from the gas composition sensor an indication of the velocity of each sound wave passing through the fuel, (ii) determine a composition of the fuel based on at least the received indication, (iii) generate a report providing an indication of one or more characteristics of the fuel based on at least the determined composition of the fuel; and (iv) transmit the report. In some embodiments, the characteristics comprise an indication of whether the fuel quality is acceptable by comparing the fuel quality to a predetermined threshold. In some embodiments, the characteristics comprise an indication of the origin of the fuel. In some embodiments, the controller is further configured to transmit the report to a user at predetermined time intervals.

According to some embodiments, a method for reporting the quality of a fuel for a vehicle is provided. The method may include, for instance, generating, by a gas composition sensor, one or more sound waves to pass through the fuel; measuring a velocity of each sound wave passing through the fuel; determining, by a controller, a composition of the fuel based on at least the measured velocity of each sound wave; generating, by the controller, a report providing an indication of the fuel quality based on at least the determined composition of the fuel; and transmitting the report. In some embodiments, the indication of the fuel quality comprises an indication of whether the fuel quality is acceptable by comparing the fuel quality to a predetermined threshold. In some embodiments, the report further comprises an indication of the origin of the fuel. In some embodiments, the transmitting is performed at predetermined time intervals based at least in part on a received control signal.

Other features and characteristics of the subject matter of this disclosure, as well as the methods of operation, functions of related elements of structure and the combination of parts, and economies of manufacture, will become more apparent upon consideration of the following description and the appended claims with reference to the accompanying drawings, all of which form a part of this specification, wherein like reference numerals designate corresponding parts in the various figures.

### BRIEF DESCRIPTION OF THE DRAWINGS

The accompanying drawings, which are incorporated herein and form part of the specification, illustrate various embodiments of the subject matter of this disclosure. In the drawings, like reference numbers indicate identical or functionally similar elements.

FIG. 1 is a cross-sectional view of an apparatus for measuring properties of a gas or fluid according to some embodiments.

FIG. 2 is a cross-sectional view of an apparatus for measuring properties of a gas or fluid according to some embodiments.

FIG. 3 is a cross-sectional view of an apparatus for measuring properties of a gas or fluid according to some embodiments.

FIG. 4 is a flow chart depicting a process for measuring properties of a gas or fluid flowing in a feed pipe according to some embodiments.

FIG. 5 is a schematic view of a system according to some embodiments.

FIG. 6 is a schematic view of a controller according to some embodiments.

FIG. 7 is a flow chart depicting a process for detecting impurities in a fuel according to some embodiments.

FIG. 8A is a schematic view of a system for determining a composition of a bio-gas comprising a methane gas and a secondary material according to some embodiments.

FIG. 8B is a schematic view of a system for determining a composition of a bio-gas comprising a methane gas and a secondary material according to some embodiments.

FIG. 9 is a schematic view of an anaerobic digester according to some embodiments.

FIG. 10 is a schematic view of a cleaning system according to some embodiments.

FIG. 11 is a flow chart depicting a process for determining a composition of a bio-gas comprising a methane gas and a secondary material according to some embodiments.

FIGS. 12A and 12B are graphs illustrating a relationship between a methane number of a fuel and a measured velocity of sound wave passing in the fuel.

FIG. 13 is a table listing the compositions of fuel mixtures according to some embodiments.

FIG. 14 is a flow chart depicting a process for reporting a quality of a fuel for a vehicle according to some embodiments.

Together with the description, the drawings further serve to explain the principles of the disclosure and to enable a person skilled in the pertinent art to make and use the embodiments disclosed herein.

### DETAILED DESCRIPTION

Natural gas comprises a hydrocarbon-based gas mixture that usually includes primarily methane and other trace gases, such as carbon monoxide, carbon dioxide, nitrogen, hydrogen sulfide, or helium. Before natural gas may be used as a fuel, and according to some embodiments, the gas mixture should be processed to remove impurities. The impurities in natural gas may comprise other forms of hydrocarbons, such as ethane, propane, butane, pentane, and hexane, which all have a greater molecular weight than the molecular weight of methane. Consequently, and according to some embodiments, the presence of impurities in a natural gas-based fuel reduces the measured speed of a sound wave passing through the fuel mixture. As the concentration of impurities increases in a natural gas-based fuel, the sound wave passes through the fuel mixture at a lower speed.

Accordingly, the composition of the fuel mixture may be analyzed by measuring a speed of a sound wave passing through the fuel mixture, thereby revealing a quality of the fuel. In particular, measuring the speed of a sound wave passing in a direction parallel to the fluid flow and in a direction opposite to the fluid flow may be used to determine one or more of a composition of the fuel mixture and the velocity of the fluid flow.

According to various embodiments, an apparatus for measuring flow properties of a gas or fluid flowing in a feed pipe may comprise a first transducer coupled to the feed pipe, a second transducer coupled to the feed pipe, and timing-circuitry in electrical communication with the first and second transducers. The first transducer is configured to generate a first sound wave passing in a first direction through the gas or fluid flowing along the feed pipe. The second transducer is configured to receive the first sound wave passing in the first direction through the gas or fluid flowing along the feed pipe. The timing-circuitry is configured to measure a velocity of the first sound wave passing in the first direction from the first transducer to the second transducer.

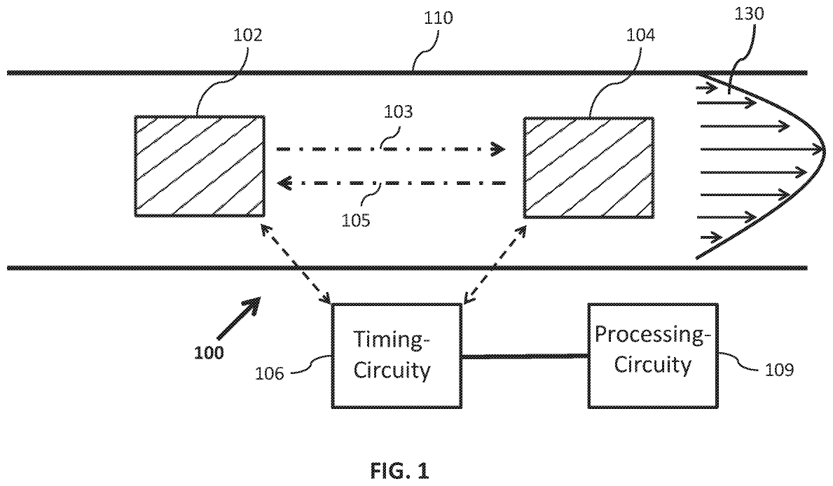

Referring to FIG. 1, an apparatus 100 for measuring properties of a gas or fluid is illustrated according to some embodiments. Apparatus 100 may, for instance, be used to evaluate the properties of a gas or fluid flowing in a feed pipe. In this example, apparatus 100 comprises a first transducer 102 coupled to a vessel, such as a feed pipe 110 in this example, at a first location, a second transducer 104 coupled to the feed pipe 110 at a second location, timing-circuitry 106 in electrical communication with the first transducer 102 and the second transducer 104, and processing-circuitry 109 in electrical communication with the timing-circuitry 106. Though illustrated as separate elements, timing-circuitry and processing-circuitry may comprise a single circuitry component. Accordingly, a single circuitry component, such as a microprocessor, may perform the functions of timing-circuitry 106 and processing-circuitry 019. Similarly, first and second transducers 102, 104 may be elements of a single sensor component. As shown in FIG. 1, in some embodiments, the first transducer 102 and second transducer 104 are disposed within a feed pipe 110 and displaced from each other by a predetermined distance along a longitudinal axis of the feed pipe 110.

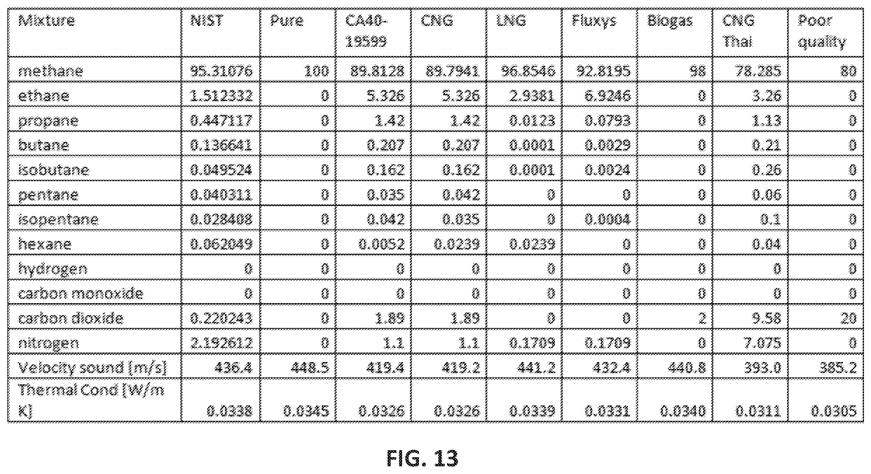

As shown in FIG. 1, a fluid or gaseous mixture 130 is flowing along the feed pipe 110. In some embodiments, the mixture 130 may include any combination of gas or liquid composition. In some embodiments, the mixture 130 may comprise a natural gas that includes a methane gas and one or more secondary components, such as, for example, carbon monoxide, carbon dioxide, nitrogen, hydrogen sulfide, helium, or other forms of hydrocarbons (e.g., ethane, propane, butane, pentane, and hexane). FIG. 13 illustrates other exemplary gas compositions for the mixture 130.

In some embodiments, the first transducer 102 and the second transducer 104 are an ultrasonic transducer configured to generate and receive an ultrasonic sound wave traveling at a frequency above, for example, 20 kHz. In some embodiments, the first transducer 102 and the second transducer 104 may comprise any combination of electrical or mechanical components such that the first and second transducers 102, 104 may generate and/or receive a sound wave. In one embodiment, the first transducer 102 and the second transducer 104 may comprise a power source (e.g., battery), a piezoelectric crystal layer, and a pair of electrodes disposed on both sides of the piezoelectric crystal layer. In this configuration, the pair of electrodes may be electrically connected to the power source such that the pair of electrodes may apply electric potential across the piezoelectric crystal layer, and in response, the piezoelectric crystal layer oscillates to generate the necessary sound wave. In some embodiments, the first transducer 102 and the second transducer 104 are each configured to convert an AC electrical signal into a sound wave by applying an electric field along the piezoelectric crystal layer so that the piezoelectric crystal layer changes in size to generate the necessary sound wave.

In some embodiments, the timing-circuitry 106 may comprise any combination of electrical components such that the timing-circuitry 106 is configured to measure a flight time of a sound wave passing between the first transducer 102 and the second transducer 104 and determine a velocity of the sound wave passing between the first transducer 102 and the second transducer 104 using least the measured flight time. In one embodiment, the velocity of the sound wave may be determined by dividing the measured flight time of the sound wave over the distance between the first location of the first transducer 102 and the second location of the second transducer 104. In some embodiments, the timing-circuitry 106 may include one or more of analog circuit components, digital circuit components, a resistive-capacitive circuit, a frequency divider, a counter, an oscillator, a processor, one or more transistors, a central processing unit (CPU), a microprocessor, a processor, a digital signal processor (DSP), a field programmable gate array (FPGA), any type of read only memory (ROM), and any type of random access memory (RAM). Timing-circuitry may comprise, for example, a raspberry pi.

In some embodiments, the processing-circuitry 109 may comprise any type of intelligent hardware device and other electrical components such that the processing-circuitry 108 is configured to receive sound wave velocity measurements from the timing-circuitry 106 and analyze the properties of the mixture 130 based on least the received velocity measurements. In some embodiments, the analysis of the properties may include determining the composition of the mixture 130 (e.g., relative number of elements making up the fluid mixture), the velocity of the mixture 130, or the thermal conductivity of the mixture 130. In some embodiments, the processing-circuitry 109 may include one or more of a CPU, a microprocessor, a controller, a microcontroller, an ASIC, a general-purpose processor, DSP, FPGA, a combination of discrete gate or transistor logic components, any type of ROM, and any type of ROM. In some embodiments, the processing-circuitry 108 and the timing-circuitry 106 may be integrated together as an integrated circuit formed on a single printed circuit board. In other embodiments, the processing-circuitry 109 may be remotely located from the timing-circuitry 106 such that the processing-circuitry 108 is in wired or wireless communication with the timing-circuitry 106. According to some embodiments, the timing-circuitry 106 and/or processing-circuitry 109 may communicate with one or more of the transducers 102, 104 over a physical or wireless connection. For example, in some embodiments, one or more of the timing-circuitry 106, the processing-circuitry 109, the first transducer 102, and the second transducer 104 include a transceiver and/or antenna to communicate wirelessly with each other.

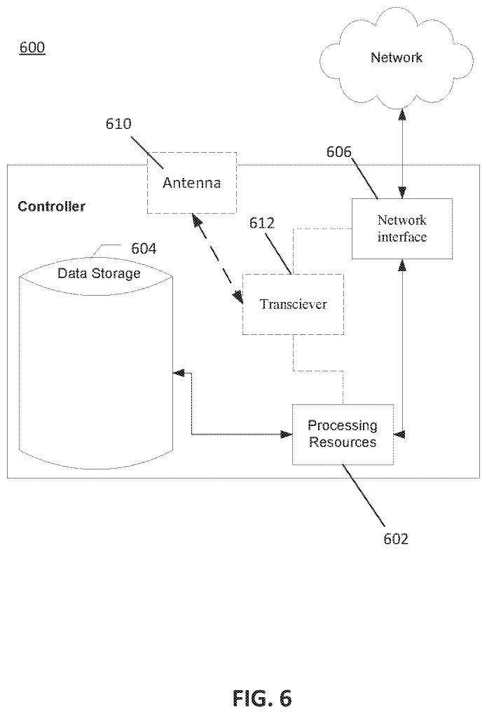

According to some embodiments, the functionality of one or more of the timing-circuitry 106, the processing-circuitry 109, the first transducer 102, and the second transducer 104 may be controlled by a controller, such as controller 600. Additionally, one or more of timing-circuitry circuitry 106, the processing-circuitry 109, the first transducer 102, and the second transducer 104 may comprise internal control functionality. This control may be, for instance, activation of the first and second transducers 102, 104 to generate the sound waves. In some embodiments, timing-circuitry 106 may further control the transducers. In some embodiments, the timing-circuitry 106, the processing-circuitry 109, and the controller 600 may be a single circuitry component, such as s microprocessor or any arrangement as described in connection with FIG. 6.

As shown in FIG. 1, the first transducer 102 is configured to generate a first sound wave 103 passing in the mixture 130 at a first direction parallel to the flow of mixture 130 flowing along the feed pipe. The second transducer 104 is configured to receive the first sound wave 103 passing in the first direction through the fluid mixture 130. The timing-circuitry 106 is configured to measure a velocity of the first sound wave 103 passing in the first direction from the first transducer 102 to the second transducer 104. Referring to FIG. 1, the second transducer 104 is configured to generate a second sound wave 105 passing in the fuel mixture 130 at a second direction opposite to the flow of the fluid mixture 130. The first transducer 102 is configured to receive the second sound wave 105 passing in the second direction through the fluid mixture 130. The timing-circuitry 106 is configured to measure a velocity of the second sound wave 105 passing in the second direction from the second transducer 104 to the first transducer 102. According to some embodiments, timing-circuitry 106 triggers the generation of the first sound wave 103 and the second sound wave 105.

In some embodiments, the timing-circuitry 106 is configured to calculate an average sound wave velocity based on at least the measured velocities of the first sound wave 103 and the second sound wave 105. In some embodiments, the average sound wave velocity may be calculated by determining the arithmetic mean of the velocity measurements or using weighted averages that take into account other factors, such as fluid or gas temperature variations, electromagnetic interference, or turbulence of fluid flow. For instance, the velocity of sound in a gas is proportional to the square root of the temperature. In some embodiments, the timing-circuitry 106 is configured to calculate the velocity of the mixture 130 by calculating the difference between the velocity of the first sound wave 103 and the velocity of the second sound wave 105. In other embodiments, the timing-circuitry 106 is configured to only measure the velocity of the sound waves 103, 105, and the processing circuitry 109 calculates the average sound wave velocity and the velocity of the mixture 130 based on least the measured velocities of the sound waves 103, 105.

According to some embodiments, the measured velocity of a sound wave is increased in the direction of flow by the fluid velocity, and the measured velocity against the flow is similarly reduced by the fluid velocity. As such, the difference between the two measured velocities is therefore twice the fluid velocity. According to some embodiments, circuitry determines the fluid velocity by calculating half of the difference between the two measured velocities. Additionally, the average of the two aforementioned measurements can give the velocity of sound as would be measured in a stationary fluid. Thus, and according to some embodiments, circuitry determines the velocity of the soundwaves based on the average of the two measured velocities.

In some embodiments, the processing circuitry 109 is configured to determine a composition of the mixture 130 based on at least the average sound wave velocity calculated by the timing-circuitry 106. In one embodiment, in which the mixture 130 is natural gas comprising primarily methane gas, the processing circuitry 109 may determine the composition of the mixture 130 by correlating the average speed of the mixture 130 to the methane number of the mixture 130.

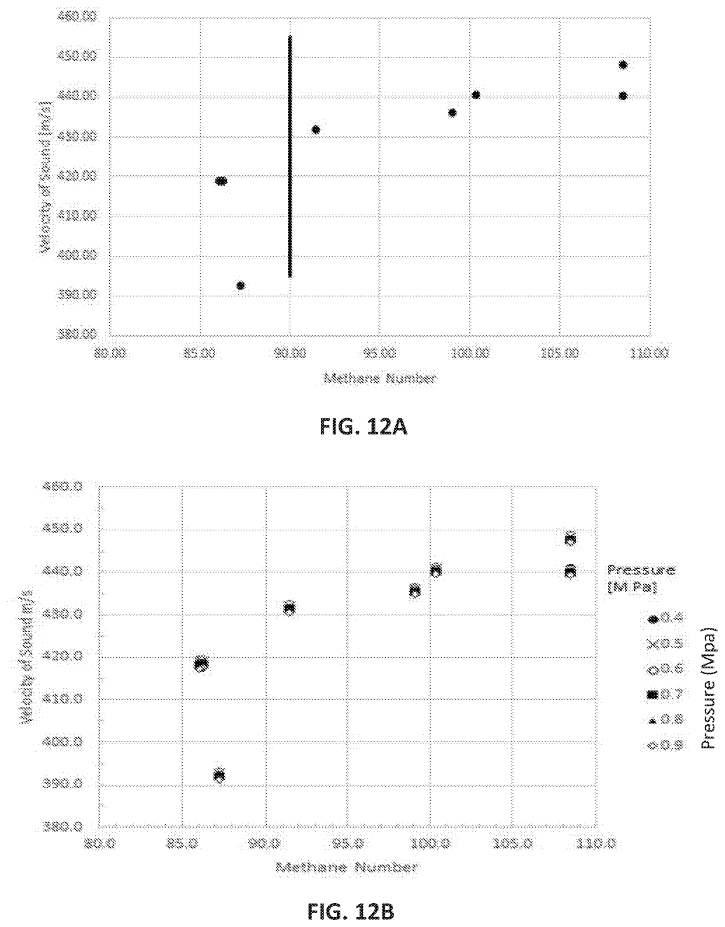

FIGS. 12A and 12B illustrate the relationship between the methane number of a gaseous or fluid mixture and the velocity of the sound wave passing through the mixture according to some embodiments. As shown in FIGS. 12A, 12B, the x-axis reflects the methane number, and the y-axis reflects the velocity of the sound wave. FIG. 12B further shows the measured velocity for each sound wave conducted over a range of pressures. Because methane gas is lighter than other trace gases and impurities found in the natural gas, a sound wave will travel at a faster rate in a mixture 130 having a greater methane number compared to a mixture 130 having a smaller methane number. As shown in FIGS. 12A and 12B, the methane number of the mixture increases as the measured velocity of the sound wave increases according to a linear relationship. A similar graph may be produced for comparing the velocity of the sound waves to the thermal conductivity of the mixture. Accordingly, the processing circuitry 109 may correlate a measured sound wave traveling at a greater speed to a mixture having a greater methane number or higher thermal conductivity, and the processing circuitry 109 may correlate a measured sound wave traveling at a slower speed to a mixture having a smaller methane number and smaller thermal conductivity. One or more of the calculated methane number and thermal conductivities of the mixture may be used to estimate the fuel quality of a fuel supplied to an internal combustion engine or estimate the quality of the bio-gas produced from anaerobic digestion process. Ultimately, the apparatus 100 may be used in fuel supply system of vehicle to prevent damage to vehicle caused by knocking, or be used in a bio-gas separation plant to that ensure an acceptably pure methane gas is achieved through filtering a bio-gas produced according to an anaerobic digestion process.

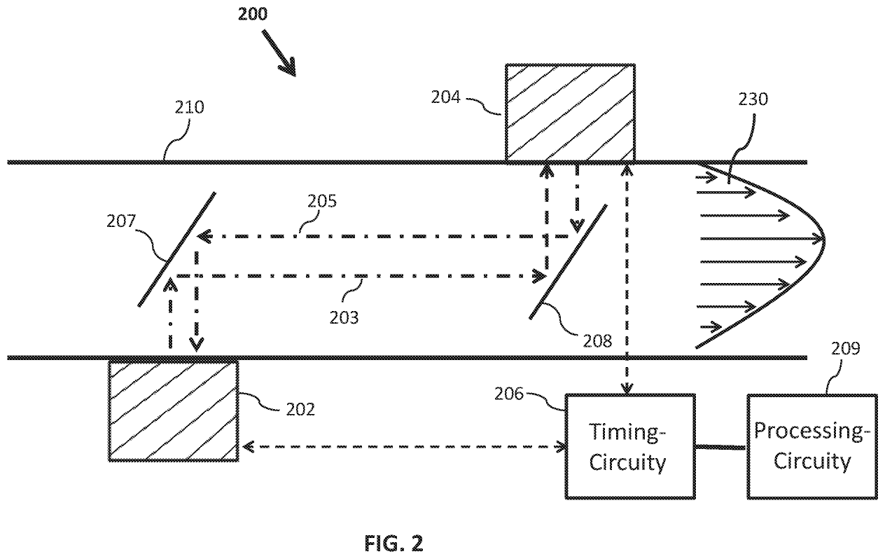

FIG. 2 illustrates an apparatus 200 for measuring flow properties of a mixture 230 flowing in a feed pipe 210 according to some embodiments. Similar to the apparatus 100 shown in FIG. 1, apparatus 200 comprises a first transducer 202 coupled to the feed pipe 210 at a first location, a second transducer 204 coupled to the feed pipe 210 at a second location, timing-circuitry 206 in electrical communication with the first transducer 202 and the second transducer 204, and processing-circuitry 209 in electrical communication with the timing circuitry 206. However, as shown in FIG. 2, the first transducer 202 and second transducer 204 are disposed on an exterior surface of the feed pipe 210 and displaced from each other by a predetermined distance along a longitudinal axis of the feed pipe 210. The apparatus 200 further comprises a first reflector 207 disposed within the feed pipe 210 and aligned with the first transducer 202 and a second reflector 208 disposed within the feed pipe 210 and aligned with the second transducer 204. According to some embodiments, feed pipe 210 is approximately 10-20 mm in width and the transducers are spaced between 100 mm and 200 mm apart. In one example, a 150 mm spacing can be used with a 15 mm pipe. Other dimensions and distance ranges may be used according to embodiments. For instance, the spacing may be reduced to adjust precision, and may be minimized with a compact sensor setup. Additionally, a similar spacing may be used in the arrangement of FIG. 1 in some embodiments.

According to some embodiments, a gas composition sensor may comprise first transducer 102, second transducer 104, and one or more of timing-circuitry 106 and processing-circuitry 109.

In some embodiments, the first reflector 207 and the second reflector 208 may comprise any combination of materials and may be formed in any shape (e.g., planar, parabolic, semi-sphere) such that the first and second reflectors 207, 208 are configured to alter the direction of a respective sound wave passing through the mixture 230, whereby the reflected sound wave is received by one of the first and second transducers 202, 204. In some embodiments, the first and second reflectors 207, 208 each comprise a flat mirror and extend obliquely with respect to the longitudinal axis of the feed pipe 210. In some embodiments, the first and second reflectors 207, 208 are mounted to the feed pipe 210 by a pedestal (not shown) comprising a first end secured to an interior surface of the feed pipe 210 and a second end secured to a respective reflector. In some embodiments, the pedestal is configured to pivot or rotate so that the orientation of the first and second reflectors 207, 208 may be adjusted accordingly with respect to the positions of the first and second transducers 202, 204.

As shown in FIG. 2, the first transducer 202 is configured to generate a first sound wave 203 passing in the fluid mixture 230 at a first direction transverse to the flow of mixture 230 flowing along the feed pipe 210. The first reflector 207 is configured to alter the direction of the first sound wave 203 so that the first sound wave 203 is aligned with the flow of the mixture 230 and directed toward the second reflector 208. The second reflector 208 is configured to alter the direction of the first sound wave 203 so that the first sound wave 203 is directed toward the second transducer 204, which is configured to receive the first sound wave 203. The second transducer 204 is configured to generate a second sound wave 205 passing in the fluid mixture 230 in a second direction transverse to the flow of mixture 230 flowing along the feed pipe 210. The second reflector 208 is configured to alter the direction of the second sound wave 205 so that the second sound wave 205 is aligned with the flow of the mixture 230 and directed toward the first reflector 207. The first reflector 207 is configured to alter the direction of the second sound wave 203 so that the first sound wave 203 is directed toward the second transducer 204, which is configured to receive the first sound wave 203. According to some embodiments, the sound waves 203 and 205 are produced sequentially. For instance, generation of the second sound wave 205 may be triggered by the receipt of the first sound wave 203. Such sequential generation may also be used in the arrangement of FIG. 1.

Referring to FIG. 2, the timing-circuitry 206 and the processing-circuitry 209 are similar to the embodiments described above. For instance, the timing-circuitry 206 can be configured to measure a velocity of the first sound wave 203 and a velocity of the second sound wave 205 and calculate an average sound wave velocity and a velocity of the mixture 230 based at least on the measured velocities of sound waves 203 and 205. The processing-circuitry 209 can be configured to determine a composition of the mixture 230 based on at least the average sound wave velocity calculated by the timing-circuitry 206.

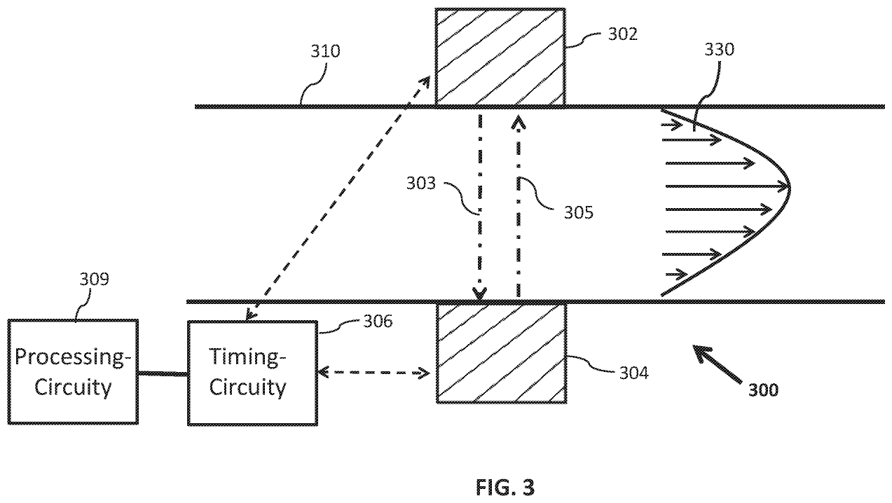

FIG. 3 illustrates an apparatus 300 for measuring the properties of a mixture 330 flowing in a vessel 310 according to some embodiments. Vessel 310 may be, for instance, a flow pipe. Similar to apparatuses 100, 200 shown in FIGS. 1 and 2, apparatus 300 comprises a first transducer 302 coupled to vessel 310 at a first location, a second transducer 304 coupled to the vessel; 310 at a second location, timing-circuitry 306 in electrical communication with the first transducer 302 and the second transducer 304, and processing-circuitry 309 in electrical communication with the timing-circuitry 306. Similar to apparatus 200 shown in FIG. 2, the first transducer 302 and second transducer 304 are disposed on an exterior surface of the feed pipe 310. However, as shown in FIG. 3, the first transducer 302 and the second transducer 304 are aligned with each other along a lateral axis of the vessel 310 in this example.

Referring to FIG. 3, the first transducer 302 is configured to generate a first sound wave 303 passing through the mixture 330 in a first direction towards a second transducer 304. According to some embodiments, second transducer 304 may merely be a receiver, such that timing-circuitry 306 and processing-circuitry 309 can determine the velocity of sound wave 303 and evaluate mixture 330 based on a single wave, or a series of waves in a single direction, from transducer 302. For example, processing-circuitry 309 may be configured to determine a composition of the mixture 330 based on at least the sound wave 303 velocity calculated by the timing-circuitry 306.

However, and according to some embodiments, the second transducer 304 may be further configured to receive the first sound wave 303 passing in the first direction through the fluid 330 and to generate a second sound wave 305 passing in the mixture 330 in a second direction opposite to the first direction. In this embodiment, the first transducer 302 is configured to receive the second sound wave 305 passing in the second direction through the mixture 330. Referring to FIG. 3, the timing-circuitry 306 and the processing-circuitry 309 are similar to the embodiments described above. The timing circuitry 306 is configured to measure a velocity of the first sound wave 303 and a velocity of the second sound wave 305 and calculate an average sound wave velocity based on at least the measured velocities of sound waves 303 and 305. The processing circuitry 309 is configured to determine a composition of the mixture 330 based on at least the average sound wave velocity calculated by the timing-circuitry 306.

Additionally, where vessel 310 is a flow pipe and transducers 302 and 304 are aligned along a lateral access of vessel 310, their position may be offset to account for the rate of flow of 330 and its effect on the sound waves 303 and 305. That is, alignment of the transducers may include an offset according to some embodiments. The amount of offset necessary will be a function of the rate of flow 330 and width of vessel 310. For instance, a great amount of offset may be needed for a higher flow rate or higher width.

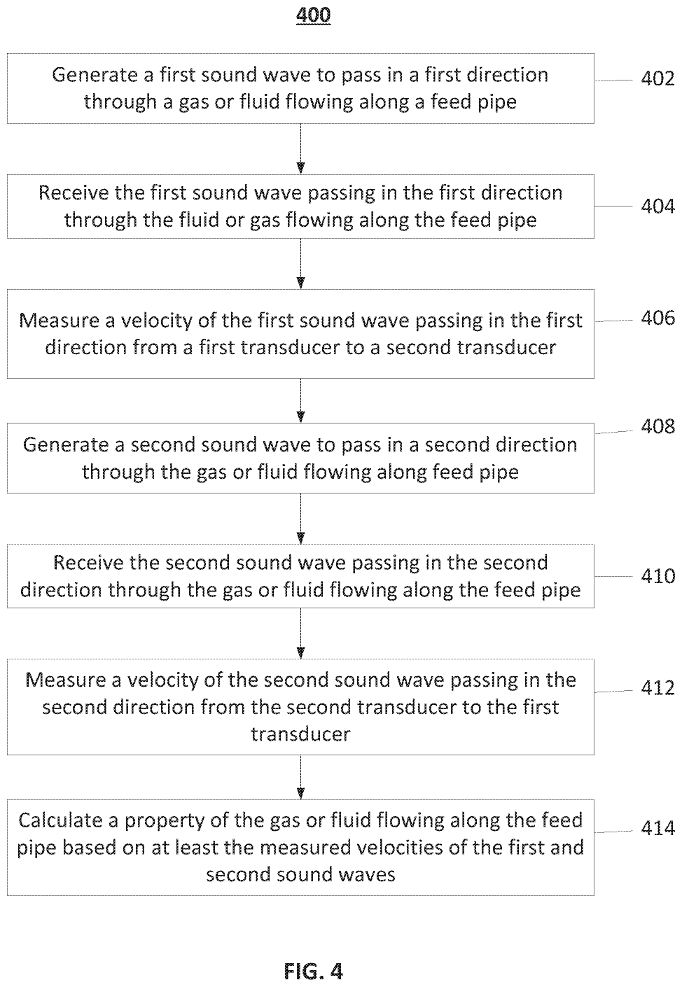

FIG. 4 is a flow chart illustrating a method 400 for measuring properties of a gaseous or fluid mixture in a feed pipe or other vessel, according to some embodiments. The method 400 may be performed by any one of the embodiments of the apparatus 100, 200, 300 described in FIGS. 1-3. As shown in FIG. 4, the method 400 may begin at step 402 with generating, by a first transducer, a first sound wave to pass in a first direction through a gas or fluid, such as a mixture flowing in a feed pipe. In some embodiments, the first sound wave may travel in a direction parallel to the longitudinal axis of the feed pipe or in a direction transverse to the longitudinal axis of the feed pipe. In some embodiments, the direction of the first sound wave may be altered by one or more reflectors disposed within the feed pipe.

According to embodiments, method 400 further comprises a step 404 of receiving, by a second transducer, the first sound wave passing in the first direction through the mixture, for instance, along a feed pipe. In some embodiments, the first and second transducers may be disposed within a feed pipe and displaced from each other along the longitudinal axis of the feed pipe. In other embodiments, the first and second transducers may be disposed on an exterior surface of a feed pipe.

Referring to FIG. 4, method 400 may further comprise a step 406 of measuring, by timing-circuitry, a velocity of the first sound wave passing in the first direction from the first transducer to the second transducer, and a step 408 of generating, by the second transducer, a second sound wave to pass in a second direction through the mixture. In some embodiments, the second sound wave may travel in a direction parallel to the longitudinal axis of a feed pipe or in a direction transverse to the longitudinal axis of a feed pipe. In some embodiments, the direction of the second sound wave may be altered by one or more reflectors disposed within a feed pipe.

Method 400 may further comprise a step 410 of receiving, by the first transducer, the second sound wave passing in the second direction through the mixture, and a step 412 of measuring, for instance by timing-circuitry, a velocity of the second sound wave passing in the second direction from the second transducer to the first transducer. Method 400 may also comprise calculating, by the timing-circuitry, an average sound wave velocity based on at least the measured velocities of the first sound wave and the second wave. In step 414, a property of the gas or fluid may be evaluated. For instance, the velocity or composition of a gas or fluid flowing along a feed pipe may be calculated based on one or more of the first, second, and/or average sound wave velocities.

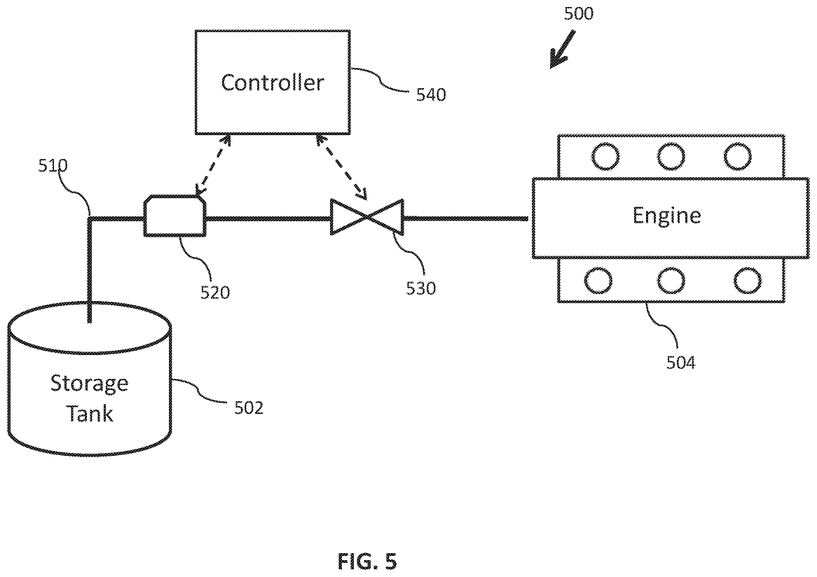

Referring to FIG. 5, a system 500 for detecting impurities in a fuel comprising methane gas according to some embodiments is illustrated. System 500 may comprise, for example, a storage tank 502 configured to store the fuel, an engine 504 configured to operate on the fuel, and a feed pipe 510 configured to supply the fuel from the storage tank 502 to the engine 504. In some embodiments, the storage tank 502 may comprise any type of container such that the storage tank 502 is configured to hold the fuel and vent the fuel to the feed pipe 510. In some embodiments, the engine 504 may comprise any type of engine (e.g., compression-ignition, spark-ignition) with any type of fuel injection system (e.g., pump-line nozzle, unit injector, and common rail systems) such that the engine 504 is configured to convert chemical energy stored in the fuel to mechanical energy.

As shown in FIG. 5, the system 500 further comprises a gas composition sensor 520 coupled to the feed pipe 510, a flow control valve 530 coupled to the feed pipe and disposed downstream of the gas composition sensor 520, and a controller 540 in electrical communication with the gas composition sensor 520 and/or the flow control valve 530. In some embodiments, the gas composition sensor 520 is configured to generate a sound wave passing through the fuel in the feed pipe 510 and measure a velocity of the sound wave passing through the fuel in the feed pipe 510, for instance, as discussed with respect to FIGS. 1-4. In some embodiments, the flow control valve 530 may comprise any type of valve (e.g., solenoid, rotary, butterfly, diaphragm, ball valve) such that the flow control valve 530 is configured to increase or reduce a flow rate of the fuel flowing along the feed pipe 510 to the engine 504.

In some embodiments, the gas composition sensor 520 may comprise any one of the embodiments of the apparatus 100-300 described herein, in which the gas composition sensor comprises a first transducer coupled to the feed pipe 510, a second transducer coupled to the feed pipe 510, and timing-circuitry and/or processing-circuitry in electrical communication with the first and second transducers. In some embodiments, the first and second transducers are disposed within the feed pipe 510 and displaced from each along a longitudinal axis of the feed pipe 510. In some embodiments, the first and second transducers are disposed on an exterior surface of the feed pipe 510 and displaced from each along a longitudinal axis of the feed pipe 510. In some embodiments, the first and second transducers are disposed on an exterior surface of the feed pipe 510 and aligned with each other along a lateral axis of the feed pipe 510.

According to certain embodiments, the controller 540 can be configured to monitor aspects of the system 500, including the quality of the fuel in the storage tank 502 and the feed pipe 510. For example, in some embodiments, the controller 540 is configured to receive an indication of the velocity of one or more sound waves passing through the fuel from the gas composition sensor 520 or a direct indication of the fuel quality based on such sound waves. In some embodiments, the indication of the velocity of one or more sound waves or quality may be transmitted through a wired connection between the gas composition sensor 520 and the controller 540. In some embodiments, the indication of the velocity of the one or more sound waves or quality may be transmitted from the gas composition sensor 520 to the controller 540 by wireless communication (e.g., Near Field Communication, Bluetooth, Local Area Network, Wide Area Network). In some embodiments, the controller 540 is further configured to receive an indication of the temperature of the fluid in the fuel by one or more temperature sensors (e.g. thermocouple).

In some embodiments, the controller 540 is configured to determine a composition of the fuel based on at least the received indication. In some embodiments, the received indication of the velocity of the sound waves includes an average velocity of the sound waves based on two or more sound waves generated by the composition sensor 520. In some embodiments, the controller 540 is configured to calculate an average sound wave velocity based on the measured sound waves found in the indication signal from the sensor. In some embodiments, similar to the processing-circuitry described elsewhere herein, the controller 540 may determine the composition of the fuel by correlating the average speed of the sound wave to the methane number of the fuel. Additionally, controller 540 may be configured to correlate the speed of a single sound wave to the methane number of the fuel. In some embodiments, the received indication may include one or more temperature measurements of the fuel. In some embodiments, the controller 540 is configured to correlate the temperature measurements of the fuel to the methane number of the fuel.

In some embodiments, the controller 540 is configured transmit a command to the flow control valve 530 to reduce the flow rate of the fuel flowing along the feed pipe 510 to the engine 502 when the level of methane is below a predetermined threshold. That is, controller 540 may control valve 530 based on a determination of the composition of the fuel. In some embodiments, the predetermine threshold is a methane number of 90.

In some embodiments, the controller 540 may be configured to communicate directly with engine 504 to adjust one or more properties of the engine including the fuel injection timing, the volume of injected fuel, and air-to-fuel ratio. For example, the speed of the sound wave (and related fuel properties) can correlate to the time required for the pressure to rise at an injection nozzle of a piston of the engine. In some embodiments, the controller 540 may transmit a command to adjust the timing of the fuel injection based on a calculated time required for the pressure to rise at the injection nozzle. The controller 540 may also transmit a command to control the volume of injected fuel or to adjust the ratio of air-to-fuel based on measured the calculated quality of the fuel or a flow property.

FIG. 6 illustrates a block diagram of a controller 600. This could be, for instance, the controller 540 in system 500 according to some embodiments. As shown in FIG. 6, a controller 600 may include: a data processing system 602, which may include one or more data processing devices each having one or more microprocessors and/or one or more circuits, such as an application specific integrated circuit (ASIC), Field-programmable gate arrays (FPGAs), etc.; a data storage system 604, which may include one or more computer-readable mediums, such as non-volatile storage devices and/or volatile storage devices (e.g., random access memory (RAM)); and a network interface 606 for connecting controller 608 to a network (e.g., an Internet Protocol (IP) network). The controller 600 may communicate with the gas composition sensor 520, the flow control valve 530, and engine 504 via the network connection. In some embodiments, the controller 600 may include a transceiver 612 and antenna 610 to communicate wirelessly with the gas composition sensor 520, the flow control valve 530, and the engine 504. According to some embodiments, controller 600 has Internet of Things (IoT) communication and control capability, for instance, through one or more of its processing resources 602, transceiver 612, antenna 610, and network interface 606. For instance, controller 600 may perform monitoring and/or measurements, and report its results to one or more other network nodes, a central server, and/or an information subscriber. Similarly, controller 600 may be configured to receive information and control signals via an IoT connection, for instance, through one or more of its processing resources 602, transceiver 612, antenna 610, and network interface 606.

In embodiments where data processing system 602 includes a microprocessor, a computer program product is provided, which computer program product includes: computer readable program code (software), which implements a computer program, stored on a computer readable medium, such as, but not limited, to magnetic media (e.g., a hard disk), optical media (e.g., a DVD), memory devices (e.g., random access memory), etc. In some embodiments, computer readable program code is configured such that, when executed by data processing system 602, the code causes the controller to perform the steps described herein (e.g., one or more steps shown in the flowcharts and/or described in connection with FIGS. 4, 7, 11, and 14). In other embodiments, controller 600 may be configured to perform steps described herein without the need for additional code. For example, data processing system 602 may consist merely of specialized hardware, such as one or more application-specific integrated circuits (ASICs). Hence, the features of the present disclosure described above may be implemented in hardware and/or software. According to some embodiments, a controller 600 may perform the steps described with respect to timing-circuitry 106 and/or processing-circuitry 109.

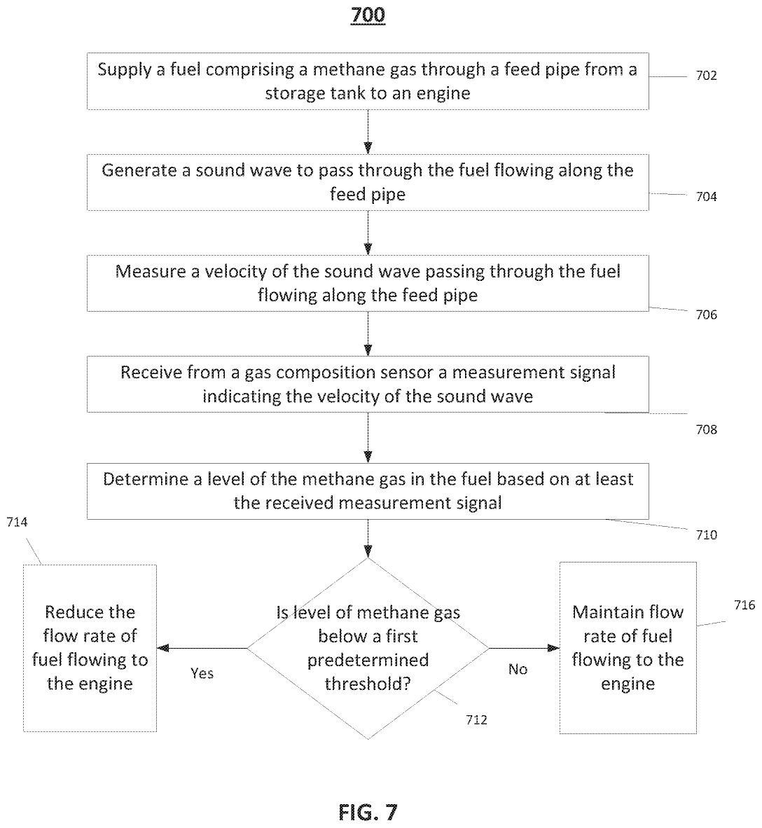

FIG. 7 is a flow chart illustrating a method 700 for detecting impurities in a fuel comprising a methane gas according to some embodiments. The method 700 may be performed, for example, by the components in the system 500 described in FIGS. 5 and 6. As shown in FIG. 7, the method 700 comprises a step 702 of supplying, by the feed pipe 510, the fuel from the storage tank 502 to the engine 504. In some embodiments, step 702 includes using a pump or pressure differential to drive the fuel from the storage tank 502 to the engine 504.

In some embodiments, the method 700 comprises a step 704 of generating, by a gas composition sensor 520, one or more sound waves to pass through the fuel flowing along the feed pipe 510 to the engine 504. In some embodiments, step 704 includes generating, by a first transducer of the gas composition sensor 520, a first sound wave to pass through the fuel in the feed pipe 510, and receiving, by a second transducer of the gas composition sensor 520, the first sound wave. In some embodiments, step 704 includes generating, by the second transducer of the gas composition sensor 520, a second sound wave to pass through the fuel in the feed pipe 510, and receiving, by the first transducer of the gas composition sensor 520, the second sound wave. However, in some embodiments, for instance with respect to the arrangement of FIG. 3, a single sound wave, or a series of sound waves in a single direction from the first transducer to a receiver, can be used.

In some embodiments, the method 700 comprises a step 706 of measuring, by the gas composition sensor 520, a velocity of the sound wave passing through the fuel flowing along the feed pipe 510 to the engine 504. In some embodiments, step 706 includes using a timing-circuitry to measure the velocity of the sound wave. In some embodiments, step 706 includes measuring the speed of a first sound wave passing from the first transducer to the second transducer and the speed of the second sound wave passing from the second transducer to the first transducer. In some embodiments, step 706 includes calculating an average sound wave velocity based on the measured speed of the first and second sound waves. In embodiments where a single sound wave is used, the velocity of that wave may be directly measured.

In some embodiments, the method 700 comprises a step 708 of receiving, by the controller 540, from the gas composition sensor 520 a measurement signal indicating the velocity of the one or more sound waves. In some embodiments, the measurement signal indicates a measured speed of a first sound wave passing from the first transducer to the second transducer and the second sound wave passing from the second transducer to the first transducer. In some embodiments, the measurement signal includes the average sound wave velocity based on the measured speed of the first and second sound waves. According to some embodiments, step 708 may be optional. For instance, signal measurement and methane level determinations may be made in the gas composition sensor.

In some embodiments, the method 700 comprises a step 710 of determining, by the controller 540, a level of the methane gas in the fuel based on at least the received measurement signal. In some embodiments, step 710 includes correlating the average speed of the sound wave passing in the fuel to the methane number of the fuel. For embodiments using a single sound wave, step 710 may include correlating the speed of the wave in the fuel to the methane number of the fuel. Additionally, and in some embodiments, the determining 710 may be performed directly by the sensor 520.

In some embodiments, the method 700 comprises a step 712 of determining, by the controller 540, if a level of the methane gas in the fuel is below a first predetermined threshold. In some embodiments, the first predetermined threshold is a methane number of 90. If the level of methane gas determined from step 710 is below the first predetermined threshold, the method 700 includes the step 714 of reducing the flow rate of fuel flowing from the storage tank 502 to the engine 502, or shutting it off completely. In some embodiments, step 714 may include transmitting, by the controller 540, a command to the flow control valve 530. In some embodiments, step 714 may include determining, by the controller 540, if the level of methane gas determined from step 710 is below a second predetermined threshold number. In some embodiments, the second predetermined threshold is a methane number smaller than the methane number of the first predetermined threshold. If the level of methane gas determined from step 710 is below the second predetermined threshold, step 714 may further include shutting off, by the flow control valve 530, a flow of the fuel to the engine 504. In some embodiments, if a methane level is insufficient, controller 540 may communicate directly with engine 504 to alter one or more parameters of the engine's function.

If the level of methane gas determined from step 710 is above the first predetermined threshold, the method 700 may comprise the step 716 of maintaining the flow rate of fuel flowing from the storage tank 502 to the engine 504. In some embodiments, step 714 may include transmitting, by the controller 540, a command to the flow control valve 530 or updating a status log.

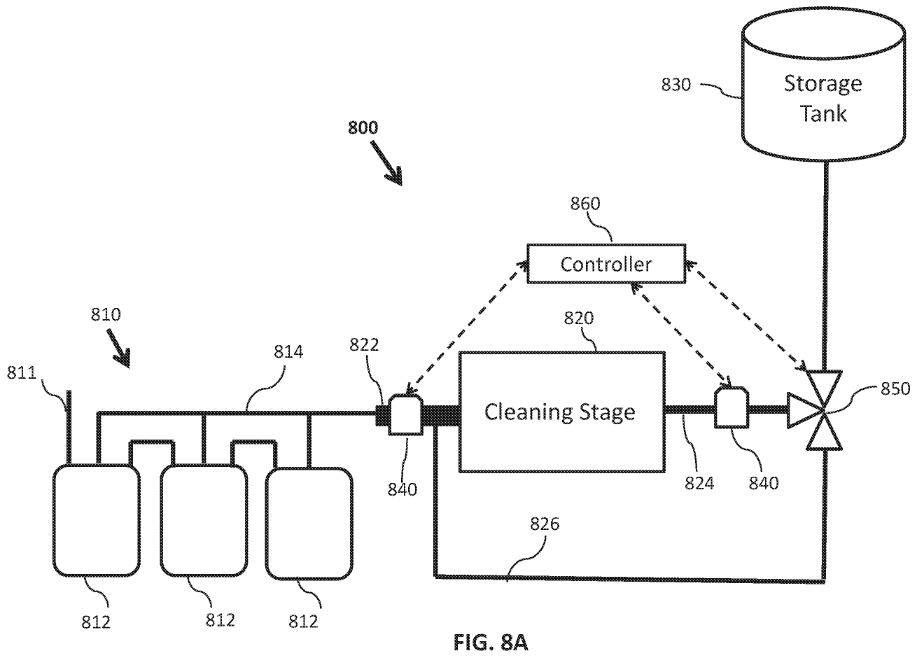

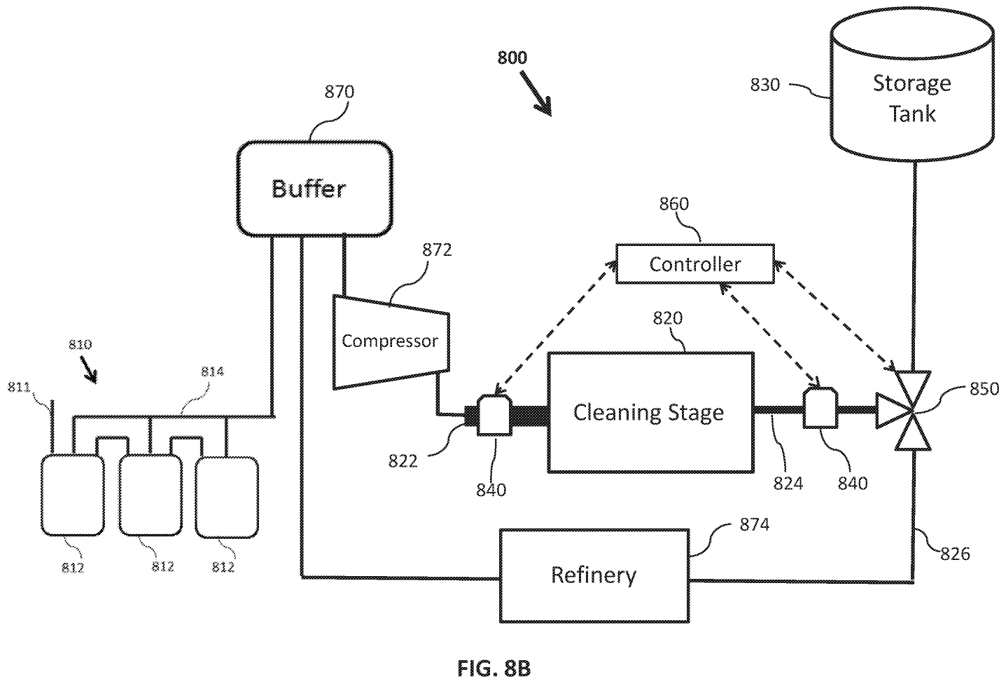

Referring to FIG. 8A, a system 800 for determining a composition of a bio-gas comprising a methane gas and a secondary material is disclosed according to some embodiments. The secondary material may be, for example, carbon dioxide. In other embodiments, the secondary material may include two or more gases or other materials, such as, for example, carbon dioxide and hydrogen sulfide. As shown in FIG. 8A, the system 800 comprises an anaerobic digestion unit 810, a gas cleaning stage 820, a storage tank 830, one or more gas composition sensors 840, a flow control valve 850, and a controller 860 in electrical communication with the gas composition sensor 840 and the flow control valve 850.

In some embodiments, the anaerobic digestion unit 810 is configured to produce the bio-gas according to an anaerobic digestion process. As shown in FIG. 8A, the anaerobic digestion unit 810 comprises one or more tanks 812 configured to vent the bio-gas and a gas outlet pipe 814 configured to receive the bio-gas vented from the plurality of storage tanks 812. In some embodiments, the anaerobic digestion unit 810 includes an inlet 811 connected to one of the tanks 812. The inlet 811 is configured to receive a biological material (e.g., feedstock, grass cuttings) and direct the biological material to the tanks 812, wherein the anaerobic digestion process is executed to produce the bio-gas. In some embodiments, the gas outlet pipe 814 is connected to each tank 812 and extends toward the gas cleaning stage 820 so that the gas outlet pipe 814 may receive the bio-gas vented from each of the tanks 812 and convey the bio-gas to the gas cleaning stage 820. Single-tank anaerobic digesters may be used according to embodiments.

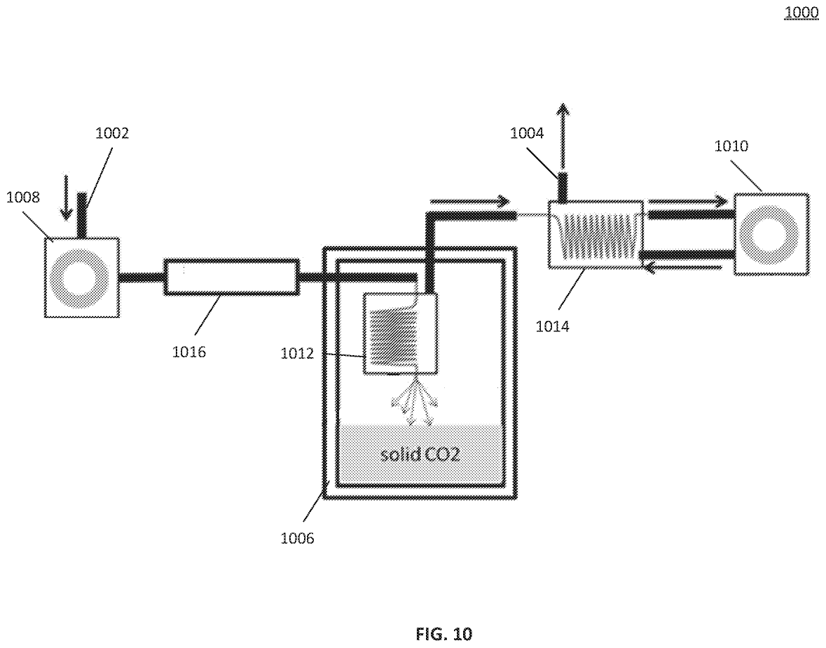

In some embodiments, the gas cleaning stage 820 is configured to remove at least a portion of the secondary material from the bio-gas produced from the anaerobic digestion unit 810. In some embodiments, the gas cleaning stage 820 comprises an inlet port 822 configured to receive the bio-gas from the outlet pipe 814 and an outlet port 824 configured to supply a remaining portion of the bio-gas to the methane storage tank 830. In some embodiments, the inlet port 822 and the outlet port 824 may include a one or more of pipes, pipe fittings, and manifolds such that the inlet and outlet ports 822, 824 may receive and direct the bio-gas. In some embodiments, the gas cleaning stage 820 comprises a storage unit (see FIG. 10) connected to the inlet port 822 and the outlet port 824, and the storage unit is configured to store a portion of the secondary material removed from the biogas and vent a remaining portion of the bio-gas toward the outlet port 824. In some embodiments, the gas cleaning stage 820 comprises a Dewar assembly such that the gas cleaning stage 820 is configured to separate liquid methane from solid carbon-dioxide.

As shown in FIG. 8A, in some embodiments, a first gas composition sensor 840 is coupled to the outlet port 824 of the gas cleaning stage and a second gas composition sensor 840 is coupled to the inlet port 822 of the gas cleaning stage 820. However, in other embodiments, the system 800 comprises only one gas composition sensor 840, which may be coupled to one of the inlet port 822 or the outlet port 824. In some embodiments, the gas composition sensor 840 is configured to generate a sound wave passing through the bio-gas flowing along the inlet or outlet ports 822, 824 and measure a speed of the sound wave passing through the bio-gas flowing along the inlet or outlet ports 822, 824. The measured speed may then be used, for instance in connection with controller 860, to evaluate the bio-gas. For instance, to determine relative quantities of first (e.g. methane) and second (e.g., carbon dioxide) materials in the bio-gas.

In some embodiments, the gas composition sensor 840 may comprise any one of the embodiments of the apparatus 100-300 described herein, in which the gas composition sensor comprises a first transducer coupled to the inlet or outlet ports 822, 824, a second transducer coupled to the inlet or outlet ports 822, 824, and timing-circuitry in electrical communication with the first and second transducers. In some embodiments, the first and second transducers are disposed within the inlet or outlet ports 822, 824 and displaced from each along a longitudinal axis of the inlet or outlet ports 822, 824. In some embodiments, the first and second transducers are disposed on an exterior surface of the inlet or outlet ports 822, 824 and displaced from each along a longitudinal axis of the inlet or outlet ports 822, 824. In some embodiments, the first and second transducers are disposed on an exterior surface of the inlet or outlet ports 822, 824 and aligned with each other along a lateral axis of the inlet or outlet ports 822, 824.

In some embodiments, the flow control valve 850 is configured to receive the remaining portion of the bio-gas from the outlet port 824 and selectively direct the remaining portion of the bio-gas to the methane storage tank 830 or back to the inlet port 822 of the gas cleaning stage 820, for example, if the level of a secondary material (e.g., carbon dioxide) in the bio-gas is too high. As shown in FIG. 8A, the flow control valve 850 is connected to the gas outlet port 824 and connected to a return line 826 that is connected to the inlet port 822. The flow control valve 850 is also connected to another flow line extending to the storage tank 830. In the illustrated embodiments, the flow control valve 850 is three-way valve comprising three separate ports. In other embodiments (not shown), the flow control valve may comprise two two-way valves, in which each two-way valve is connected to the outlet port 824 of the gas cleaning stage. In some embodiments, the flow control valve 850 may comprise any type of valve component (e.g., solenoid, rotary, butterfly, diaphragm, ball valve) such that the flow control valve 530 is configured to selectively direct flow back to the inlet port 822 of the gas cleaning stage or to the methane storage tank 830.

In some embodiments, the controller 860 is configured to receive from the gas composition sensor 840 a measurement signal indicating the speed of the sound wave passing in the bio-gas, and determine a level of the secondary material or a methane gas level in the bio-gas passing through either of the inlet and outlet ports 822, 824 based on at least the received measurement signal. In some embodiments, the controller 860 is similar to the embodiments of the controller described in FIGS. 5 and 6 herein. In some embodiments, the controller 860 is configured to determine the level of the secondary material by correlating the average speed of the sound wave to the methane number of the bio-gas. In some embodiments, the controller 860 is configured to transmit a command to the flow control valve to direct the remaining portion of the bio-gas back to the inlet of the gas cleaning stage when the level of methane gas in the remaining portion of the bio-gas flowing along the outlet port 824 is below a predetermined threshold. In some embodiments, the predetermined threshold is a bio-gas comprising more than 99% by weight methane gas.

In some embodiments, the controller 860 may receive an indication of methane or secondary material level from the 840, which is based on a sound wave measurement. That is, in certain aspects, sensor 840 may comprise circuitry sufficient to evaluate the bio-gas and communicate the result to the controller 860. The controller 860 may then transmit its respective commands based on the evaluation received from sensor 840. In this respect, and according to embodiments, the ultimate determination of methane and/or secondary material based on sound waves measurement may be performed by a sensor or a controller.

In some embodiments, the controller 860 is configured to determine a level of the methane gas in the bio-gas passing through the inlet port 822 of the gas cleaning stage 820 based on at least a received measurement signal transmitted from the second gas composition sensor 840. In some embodiments, the controller 860 is configured to determine an efficiency of the gas cleaning stage 820 by comparing the level of methane gas in the bio-gas passing through the inlet port 822 of the gas cleaning stage 820 compared to the level of methane in the remaining portion of bio-gas passing through the outlet port 824 of the gas cleaning stage 820. If the efficiency of the gas cleaning stage 820 is insufficient, service or maintenance on the gas cleaning stage may be requested or an alarm may be triggered.

FIG. 8B illustrates an alternative embodiment of the system 800 comprising all the elements shown in FIG. 8A and further comprising a buffer 870, a compressor 872, and a refinery 874. In this embodiment, rather than sending bio-gas generated by the tanks 812 of the anaerobic digestion unit 810 directly to the inlet port 822 of the gas cleaning stage 820, the gas outlet pipe 814 directs the bio-gas to the buffer 870. The bio-gas may be stored in the buffer 870 until a predetermined pressure is reached. The compressor 872 is connected downstream of the buffer 870 and upstream of the gas cleaning stage 820. In some embodiments, the compressor 872 is configured to increase the pressure of the bio-gas to a predetermined pressure level (e.g., 250 Bar) before the bio-gas enters the gas cleaning stage 820. In some embodiments, the refinery 874 is disposed along the return line 826. In some embodiments, the refinery 874 is Joule-Thomson cooler configured to turn high pressure methane in the bio-gas into a liquid methane and route the liquid methane to a storage tank (not shown) and the remaining portion of the bio-gas back to the inlet port 822 of the gas cleaning stage 820, or alternatively, to the buffer 870.

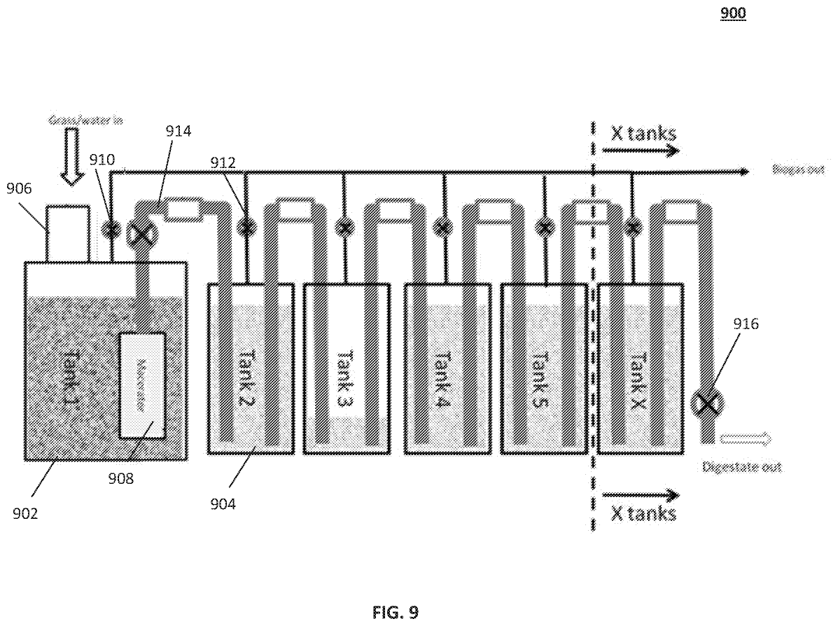

One exemplary embodiment of the anaerobic digestion (AD) unit 810 is shown in FIG. 9. The anaerobic digester of FIG. 9 may be a part of micro-AD and coupled to the system 800 shown in FIG. 8A. The anaerobic digester 900 in this example comprises multiple tanks (902,904). The total number of tanks may be set according to a customers intended use and/or the amount of land that will be used to supply feedstock. In certain aspects, a PH gradient and temperature gradient can be maintained across the tanks. The digester 900 may further include an inlet 906 for receiving feedstock and a macerator 908 to mulch, agitate and/or separate components of the feedstock during anaerobic digestion.

In some embodiments, the gas output from each tank is controlled via a latching gas valve 910,912. In certain aspects, the valve may be remotely controllable, for instance, via local or remote computer. If a quantity of substrate (e.g., partially digested feedstock) is required to be moved from one tank to the next, for instance, from tank 902 to tank 904, the gas output of the sending tank 902 can be turned off using gas valve 910. However, the gas output valve 912 of the receiving tank 904 is left open. The gas pressure in the sending tank 902 is then allowed to build up and as a result the substrate is forced though the outlet pipe 914 and into the receiving tank 904. Once the substrate move has taken place, the gas pressure from the sending tank 902 is relieved to a point at which transfer stops. The gas pressure may then be maintained at this level to prevent re-syphoning of the substrate. In some embodiments, the pressure can be completely released to allow the levels of the tanks 902,904 to re-equalize.

According to some embodiments, anaerobic digestion may be performed using multiple tanks. Typically, anaerobic digestion requires the use of heat to initiate the process. Methanogens, which are microbes that digest feedstock such as grass, can be split into two categories based on the temperature ranges at which they function. These categories/ranges are referred to as “thermophilic” p(approximately 45-70 C) and “mesophilic” (approximately 15-40 C). The thermophilic anaerobes are typically considered more difficult to sustain in a continuous process, although possible. Thermophilic anaerobes, however, are able to digest grass at a much faster rate (approximately twice as fast as mesophilic anaerobes) and can be sustained in a continuous process. In certain aspects, systems disclosed herein may be designed to operate between these two modes. For instance, a first thermophilic process can be used to break down as much of the feedstock (e.g., grass) as possible, while the remaining organic matter is then passed onto one or more additional tanks to finish of the digestion using a mesophilic process.

By controlling the generation and release of bio-gas as set forth above, and in accordance with some embodiments, the bio-gas generation and release may be synchronized with the evaluation and cleaning of bio-gas. That is, additional bio-gas may be generated on-demand when previously generated bio-gas is determined to be of sufficient quality/composition. Accordingly, the controller 860 may transmit a command to increase the output of the anaerobic digestion unit 810 when determining that the generated bio-gas is of sufficient quality/composition based on measurements received from the gas composition sensor 840.

One exemplary embodiment of the gas cleaning stage 1000 is shown in FIG. 10. The gas cleaning stage 1000 includes an inlet 1002, and outlet 1004, and a CO₂ storage unit 1006. The gas cleaning stage 1000 further includes a number of compressors (1008,1010) and heat exchangers (1012,1014), as well as an optional hydrogen sulphide filter 1016. The inlet 1002 is connected to a biogas source. For instance, inlet 1002 may be connected to anaerobic digester 810 illustrated in FIGS. 8A, 8B to receive the biogas generated by the digester 810. Outlet 1004 may be coupled to a methane storage system, such as the methane storage tank 830.

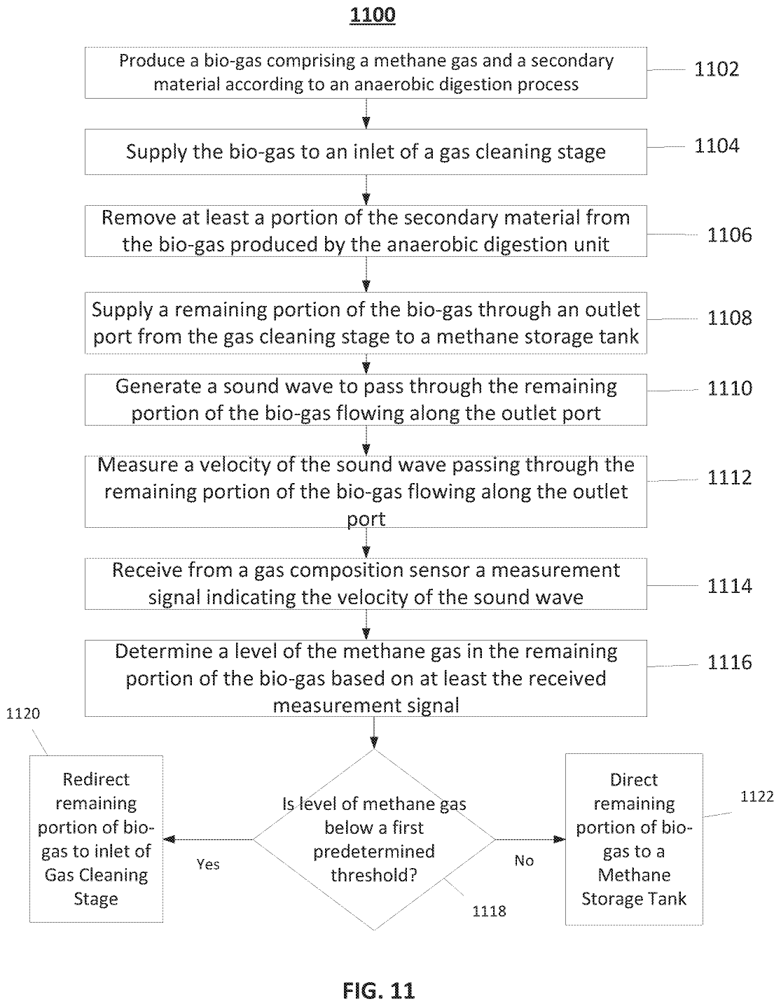

FIG. 11 is a flow chart illustrating a method 1100 for determining a composition of a bio-gas comprising a methane gas and a secondary material according to some embodiments. In some embodiments, the method 1100 may be performed by the components in the system 800 described in FIGS. 8-10. As shown in FIG. 11, the method 1100 comprises a step 1102 of producing, by an anaerobic digestion unit, such as unit 810, the bio-gas according to an anaerobic digestion process. In some embodiments, step 1102 includes supplying a biological material (e.g., feedstock) to one or more tanks 812 of the anaerobic digestion unit 810 and the bio-gas is produced using the supplied biological material.

In some embodiments, the method 1100 comprises a step 1104 of supplying, by the gas outlet pipe 814, the bio-gas gas to the inlet port 822 of the gas cleaning stage 820. In some embodiments, step 1104 may include first directing the bio-gas from the gas outlet pipe 814 to a buffer 870 before conveying the bio-gas to the gas cleaning stage 820 to increase the pressure of the bio-gas.

In some embodiments, the method 1100 comprises a step 1106 of removing, by the gas cleaning stage 820, at least a portion of the secondary material from the bio-gas produced by the anaerobic digestion unit 810. In some embodiments, step 1106 includes using a Dewar assembly (or similar storage device) to store extracted carbon dioxide, as the secondary material, from the bio-gas. In some embodiments, the extracted carbon dioxide may be in the form of a gas, solid, or a liquid.

In some embodiments, the method 1100 comprises a step 1108 of supplying, by the outlet port 824, a remaining portion of the bio-gas from the gas cleaning stage 820 to a methane gas storage tank 830.

In some embodiments, the method 1100 comprises a step 1110 of generating by a gas composition sensor 840, a sound wave to pass through the remaining portion of the bio-gas flowing along the outlet port 824. In some embodiments, step 1110 includes generating, by a first transducer of the gas composition sensor 840, a first sound wave to pass through the bio-gas in the outlet port 840, and receiving, by a second transducer of the gas composition sensor 840, the first sound wave. In some embodiments, step 1110 includes generating, by the second transducer of the gas composition sensor 840, a second sound wave to pass through the bio-gas in the outlet port 824, and receiving, by the first transducer of the gas composition sensor 840, the second sound wave.

In some embodiments, the method 1100 comprises a step 1112 of measuring, by the gas composition sensor 840, a velocity of the sound wave through the remaining portion of the bio-gas flowing along the outlet port 824. In some embodiments, step 1112 includes using timing-circuitry to measure the velocity of the sound wave. In some embodiments, step 1112 includes measuring the speed of the first sound wave passing from the first transducer to the second transducer and the speed of the second sound wave passing from the second transducer to the first transducer. In some embodiments, step 1112 includes calculating an average sound wave velocity based on the measured speed of the first and second sound waves. As described elsewhere in this disclosure, according to some embodiments, a single sound wave rather than averaged sound waves may be used.

In some embodiments, the method 1100 comprises a step 1114 of receiving, by the controller 860, from the gas composition sensor 840 a measurement signal indicating the velocity of the sound wave. In some embodiments, the measurement signal indicates a measured speed of first sound wave passing from the first transducer to the second transducer and the second sound wave passing from the second transducer to the first transducer. In some embodiments, the measurement signal includes the average sound wave velocity based on the measured speed of the first and second sound waves.

In some embodiments, the method 1100 comprises a step 1116 of determining, by the controller 860, a level of the methane gas in the remaining portion of the bio-gas based on at least the received measurement signal. In some embodiments, step 1116 includes correlating the average speed of the sound wave passing in the fuel to the methane number of the bio-gas. In some embodiments, at 1116, the methane content of the bio-gas may be determined by the sensor 840 and passed to the controller 860.

In some embodiments, the method 1100 comprises a step 1118 of determining, by the controller 860, if a level of the methane gas in the fuel is below a first predetermined threshold. Similar to step 1116, this determination may be made by the sensor and passed to the controller. In some embodiments, the first predetermined threshold is a bio-gas comprising more than 99% by weight methane gas. If the level of methane gas determined from step 1116 is below the first predetermined threshold, the method 1100 includes the step 1120 of redirecting, by the flow control valve 850 coupled to the outlet port 824, a flow of the remaining portion of the bio-gas back to the inlet port 822 of the gas cleaning stage 820 when the determined level of methane gas in the fuel is below a first predetermined threshold. In some embodiments, step 1120 may include transmitting, by the controller 860, a command to the flow control valve 850. If the level of methane gas determined from step 1116 is above the first predetermined threshold, the method 1100 includes the step 1122 of directing, by the flow control valve 850, the remaining portion of the bio-gas to the methane storage tank 830. In some embodiments, step 1122 may include transmitting, by the controller 860, a command to the flow control valve 850.

According to some embodiments, a monitoring system is provided with a gas sensor and controller, such as controller 600. The system may be provided as part of an Internet of Things (IoT) arrangement. For instance, the system may have IoT communication and control capability through one or more of terrestrial and satellite communications. In some embodiments, the system is arranged like system 500 shown in FIG. 5 in connection with an engine, such as a vehicle's engine. In certain aspects, the sensor and controller may be able to assess the nature of the origin, or otherwise asses the type, of the gas used by a vehicle. This can be in addition to other evaluations described herein.

For example, a monitoring system may be implemented in a vehicle, such as a commercial truck, and report on fuel evaluations. In some embodiments, the reports may be provided to a customer, end user, or manager of the truck. For instance, such evaluations might be important for an end user or customer of the truck company, e.g., a supermarket or clothing retail chain, that wants to ensure that only fuel that meets a level of environmental sustainability is being used. This could be, for example, to ensure that its green credentials are met. In some cases, this could be achieved if the fuel consists of a very high percentage of bio-methane versus natural gas that would have a lower methane number. Additionally, a manager of the truck may want to verify the purported source of the fuel purchased for use in the truck. Alternatively, monitoring by a customer or other end user may prevent the truck company operators from using a cheaper, inferior fuel supplier to increase profits, which potentially would harm its customers' green credentials if it were to become known. The monitoring sensor could therefore provide a customer the opportunity to independently “police” the truck operators that provide its distribution through the fitting of the monitoring sensor into their vehicles' fuel delivery systems.

According to some embodiments, the source of the fuel may be assessed based on sound wave velocity. For instance, and as illustrated with respect to FIG. 13, pure methane has a velocity of 448.5 m/s, and a poor quality fuel may have a velocity of 385.2 m/s. The monitoring system could report the measured velocity directly, report an indication of the fuel quality, report an estimate of the fuel source, or an indication of “acceptable” or “unacceptable.” In certain embodiments, the monitoring system may use a look up table, for instance, comprising information similar to that of FIG. 13, to estimate the source of the fuel, or possible sources of the fuel based on the measurements. For example, the monitoring system may be arranged to report that the fuel's source is likely “biogas” or an acceptable alternative. In some embodiments, a measured velocity below 436.4 m/s may trigger an indication of an unacceptable source. In some embodiments, a measured velocity of less than 440.8 m/s may indicate an unacceptable source. In an IoT arrangement, the monitoring system may receive updated thresholds and comparison data, for instance, from an end user or manager. In some embodiments, an end user/customer can update the acceptable/unacceptable threshold via an IoT connection.

In some embodiments, periodic measurements of the fuel are stored locally and then later reported, for instance, at pre-determined intervals. Reporting may be defined, in some instances, based on control signals received from end users/customers or a truck management facility. In some instances, a report may be requested by an end user/customer and then subsequently, in response, transmitted by the monitoring system.

In certain aspects, the monitoring sensor could be implemented entirely independently of the truck company if, for example, the fuel supply to the truck company was provided by the end customer through only pumps based at locations where accredited low carbon fuel is available. In some aspects, the monitoring system could be connected to the fuel supply source rather than on the truck or connected to an engine.

In certain aspects, the monitoring sensor may provide the means for an end user to audit its green credentials versus its competitors, which might not have taken sufficient regulatory step. This could act as a powerful marketing/branding aid for the users of trucks that adopt the sensor over they're competitors. The end customer therefore has the opportunity to not only specify the fuel used by its suppliers but also negotiate a better price for its fuel independently.

In certain aspects, for instance to save handling costs over international borders and between fuel supply companies, this could be achieved securely though the use of a cryptocurrency such as blockchain, Bitcoin etc., automatically peer-to-peer. In some embodiments, the use of the monitoring sensor in conjunction with the use of SATCOMMs reduces the possibility of the system being hacked.

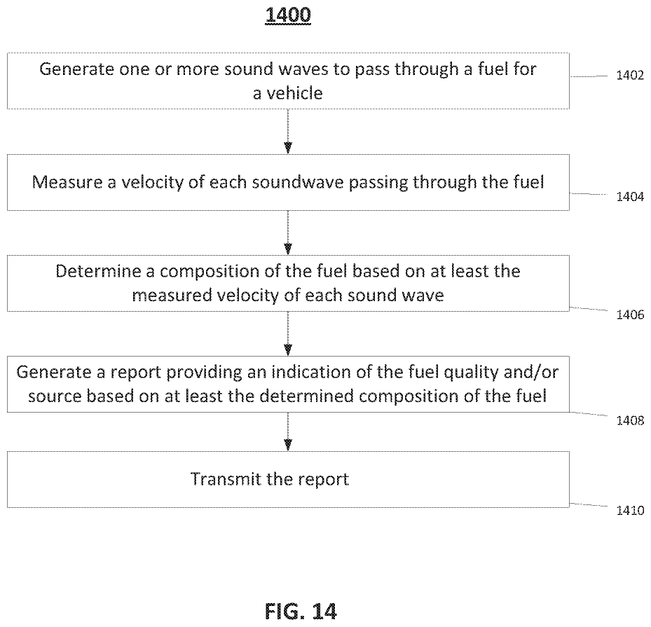

FIG. 14 is a flow chart illustrating a method 1400 for reporting fuel characteristics, such as quality and/or origin, of a fuel for a vehicle according to some embodiments. In some embodiments, the method 1400 may be performed by the components in the system 500 and controller 600 described in FIGS. 5 and 6. As shown in FIG. 14, the method 1400 comprises a step 1402 of generating, by a gas composition sensor (520), one or more sound waves to pass through the fuel for a vehicle. In some embodiments, the method 1400 comprises a step 1404 of measuring, by the gas composition sensor (520), a velocity of each soundwave passing through the fuel. In some embodiments, the method 1400 comprises a step 1406 of determining, by a controller (600), a composition of the fuel based on at least the measured velocity of each sound wave. In some embodiments, the method 1400 comprises a step 1408 of generating, by the controller (600), a report providing an indication of the fuel quality and/or its origin based on at least the determined composition of the fuel. In step 1410, the report is transmitted. The controller and sensor may be mounted on the vehicle itself. In some embodiments, the controller and sensor are mounted on a fuel source.

While various embodiments of the present disclosure are described herein, it should be understood that they have been presented by way of example only, and not limitation. Thus, the breadth and scope of the present disclosure should not be limited by any of the above-described exemplary embodiments. Moreover, any combination of the above-described elements in all possible variations thereof is encompassed by the disclosure unless otherwise indicated herein or otherwise clearly contradicted by context.

Additionally, while the processes described above and illustrated in the drawings are shown as a sequence of steps, this was done solely for the sake of illustration. Accordingly, it is contemplated that some steps may be added, some steps may be omitted, the order of the steps may be re-arranged, and some steps may be performed in parallel.

1-44. (canceled)

45. An apparatus for measuring flow properties of a gas or fluid flowing in a feed pipe, the apparatus comprising:
a first transducer coupled to the feed pipe and configured to generate a first sound wave passing in a first direction through the gas or fluid flowing along the feed pipe;
a second transducer coupled to the feed pipe and configured to receive the first sound wave passing in the first direction through the gas or fluid flowing along the feed pipe;
timing-circuitry in electrical communication with the first and second transducers and configured to measure a velocity of the first sound wave passing in the first direction from the first transducer to the second transducer.

46. The apparatus of claim 45, wherein the second transducer is configured to generate a second sound wave passing in a second direction through the gas or fluid flowing along the feed pipe, and the first transducer is configured to receive the second sound wave passing in the second direction through the gas or fluid flowing along the feed pipe, and the timing-circuitry is configured to measure a velocity of the second sound wave passing in the second direction from the second transducer to the first transducer.

47. The apparatus of claim 46, wherein the timing-circuitry is configured to calculate an average sound wave velocity based on at least the measured velocities of the first sound wave and the second wave.

48. The apparatus of claim 46, wherein the timing-circuitry is configured to calculate a velocity of the gas or fluid based on at least the measured velocities of the first sound wave and the second wave.

49. The apparatus of claim 47 further comprising processing circuitry in electrical communication with the timing-circuitry, wherein the processing-circuitry is configured to determine a composition of the gas or fluid based on at least the average sound wave velocity.

50. The apparatus of claim 45, wherein the first and second transducers are disposed within the feed pipe and are displaced from each other along a longitudinal axis of the feed pipe.

51. The apparatus of claim 45, wherein the first and second transducers are disposed on an exterior surface of the feed pipe, and the first and second transducers are displaced from each other along a longitudinal axis of the feed pipe.

52. The apparatus of claim 51 further comprising:
a first reflector disposed within the feed pipe and aligned with the first transducer;
a second reflector disposed within the feed pipe and aligned with the second transducer;
wherein the first and second reflectors are configured to alter the direction of the first and second sound waves passing through the gas or fluid to align with the direction of gas or fluid flow.

53. The apparatus of claim 52, wherein the first and second reflectors comprise a mirror.

54. The apparatus of claim 47, wherein the first and second transducers are disposed on an exterior surface of the feed pipe, and the first and second transducers are aligned with each other along a lateral axis of the feed pipe.

55. A method for measuring flow properties of a gas or fluid flowing in a feed pipe, the method comprising:
(a) generating, by a first transducer coupled to the feed pipe, a first sound wave to pass in a first direction through the gas or fluid flowing along the feed pipe;
(b) receiving, by a second transducer coupled to the feed pipe, the first sound wave passing in the first direction through the gas or fluid flowing along the feed pipe; and
(c) measuring, by timing-circuitry, a velocity of the first sound wave passing in the first direction from the first transducer to the second transducer.

56. The method of claim 55, further comprising:
(d) generating, by the second transducer, a second sound wave to pass in a second direction through the gas or fluid flowing along the feed pipe,
(e) receiving, by the first transducer, the second sound wave passing in the second direction through the gas or fluid flowing along the feed pipe, and
(f) measuring, by the timing-circuitry, a velocity of the second sound wave passing in the second direction from the second transducer to the first transducer.

57. The method of claim 56, further comprising, after step (f), calculating an average sound wave velocity based on at least the measured velocities of the first sound wave and the second wave.

58. The method of claim 56, further comprising, after step (f), calculating a velocity of the gas or fluid flowing along the feed pipe based on at least the measured velocities of the first sound wave and the second wave.

59. The method of claim 58 further comprising determining a composition of the gas or fluid based on at least the average sound wave velocity,

60. The method of claim 58, wherein the first and second transducers are disposed within the feed pipe and are displaced from each other along a longitudinal axis of the feed pipe.

61. The method of claim 58, wherein the first and second transducers are disposed on an exterior surface of the feed pipe, and the first and second transducers are displaced from each other along a longitudinal axis of the feed pipe.

62. The method of claim 61 further comprising, after step (a) and before step (b), redirecting, by a first reflector, the first sound wave toward a second reflector; and redirecting, by the second reflector, the first sound wave toward the second transducer.

63. The method of claim 62 further comprising, after step (d) and before step (e), redirecting, by the second reflector, the second sound wave toward the first reflector; and redirecting, by the first reflector, the second sound wave toward the first transducer.

64. The method of claim 62, wherein the first reflector is disposed within the feed pipe and aligned with the first transducer, and the second reflector is disposed within the feed pipe and aligned with the second transducer.

65. The method of claim 62, wherein the first and second reflectors comprise a mirror.

66. The method of claim 56, further comprising, adjusting a flow of the gas or fluid in the feed pipe or turning off an engine based at least in part on an average sound wave velocity or a calculated velocity of the gas or fluid flowing along the feed pipe.

67-76. (canceled)
